(19) United States
(12) Patent Application Publication
(10) Pub. No.: US 2025/0043065 A1
(43) Pub. Date: Feb. 6, 2025

(54) NEW ALIPHATIC POLYAMINES FOR USE AS CURING AGENT FOR EPOXY RESINS

(71) Applicant: BASF SE, Ludwigshafen Am Rhein (DE)

(72) Inventors: Martin Koenemann, Ludwigshafen (DE); Johann-Peter Melder, Boehl-Iggelheim (DE); Hannes Ferdinand Gouverneur, Ludwigshafen (DE); Radoslaw Kierat, Ludwigshafen (DE)

(73) Assignee: BASF SE, Ludwigshafen Am Rhein (DE)

(21) Appl. No.: 18/713,905

(22) PCT Filed: Nov. 21, 2022
(86) PCT No.: PCT/EP2022/082557

§ 371 Date: May 28, 2024

(30) Foreign Application Priority Data
Nov. 29, 2021 (EP) 21211116.5

(51) Int. Cl. C08G 59/50; C07C 209/52; C07C 211/14; C07D 243/08

(52) U.S. Cl. CPC C08G 59/5006; C07C 209/52; C07C 211/14; C07D 243/08

(57) ABSTRACT

Aliphatic polyamines with methyl substituents are used in the curing of epoxy resins. The preparation of such aliphatic polyamines is described. Corresponding epoxy resin compositions include an epoxy resin and the aliphatic polyamines as curing agents. These curing agents combine comparably rapid curing at moderately elevated temperatures with comparably long pot life at room temperature. At the same time, they allow for cured epoxy resins with good mechanical properties and high glass transition temperature.

The present invention relates to new aliphatic polyamines with methyl substituents for use in curing of epoxy resins as well as to the preparation of such aliphatic polyamines. These aliphatic polyamines are represented by the generic chemical formula H₂N(-Q-NH)ₙ-A—NH-Q-NH₂, with n being 0 or 1, A being —CH₂—CH(CH₃)—CH₂— or —CH₂—CH₂—CH(CH₃)— or —CH(CH₃)—CH₂—CH₂—, and each Q being independently —CH(CH₃)—CH₂— or —CH₂—CH(CH₃)—. The invention further relates to the corresponding epoxy resin compositions comprising epoxy resin and such aliphatic polyamines, the process of curing such compositions and the resulting cured epoxy resins. Such epoxy resin compositions combine comparably rapid curing at moderately elevated temperatures with comparably long pot life at room temperature. At the same time, they allow for cured epoxy resins with good mechanical properties and high glass transition temperature.

Epoxy resins are common knowledge and on account of their toughness, flexibility, adhesion and chemicals resistance are used as materials for surface coating, as adhesives and for molding and laminating as well as for producing fiber-reinforced composite materials.

Typical curing agents for epoxy resins are polyamines which bring about a polyaddition reaction (chain extension). Polyamines having a high reactivity are generally added to the epoxy resin only shortly before the desired curing. Such systems are therefore so-called two-component (2K) systems.

In principle, aminic curing agents (amino curing agents) are classified according to their chemical structure into aliphatic, cycloaliphatic or aromatic types. In addition, classification is possible by the degree of substitution of the amino group, which may be primary, secondary or else tertiary. For tertiary amines, however, a catalytic mechanism of curing of epoxy resins is postulated, whereas the basis for the formation of the polymer network for secondary and for primary amines is stoichiometric curing reactions.

In general, it has been shown that aliphatic amines show the highest reactivity among the primary amino curing agents in epoxy curing. Somewhat slower reaction is typically exhibited by cycloaliphatic amines, whereas aromatic amines (amines where the amino groups are linked directly to a carbon atom of the aromatic ring) have by far the lowest reactivity.

These known differences in reactivity are utilized in the curing of epoxy resins in order to be able to adjust the processing time and curing rate as required. In many applications such as the production of fiber-reinforced composite materials (composites), it is desirable if the freshly prepared mixture of epoxy resin and amino curing agent (epoxy resin composition) has a long processing time (pot life: period within which the composition can be processed), e.g., to allow for sufficient embedding and impregnation of reinforcing fibers. In the production of composites by means of pultrusion methods or by infusion or injection methods such as vacuum-assisted resin transfer molding (VARTM) or resin transfer molding (RTM), a sufficiently long processing time is required for the matrix component to efficiently wet the reinforcing fibers and to be distributed homogeneously around the reinforcing fibers, especially in the production of large components. For the same reason, a low mixed viscosity of the epoxy resin composition is also desirable. At the same time, the epoxy resin composition is to cure within an acceptable period of time at elevated temperature in order to enable short production cycles and hence high productivity.

Cycloaliphatic amines, for example isophoronediamine (IPDA), enable a comparatively long processing time and, given suitable formulation, simultaneously also a high curing rate and low mixed viscosity (Ullmann's Encyclopedia of Industrial Chemistry, Wiley-VCH, Weinheim, Germany, 2012, Vol. 13, Epoxy Resins, H. Pham & M. Marks, chpt. 15.1.1.2, Tab. 14 (online: 15.10.2005, DOI: 10.1002/14356007.a09_547.pub2)). Moreover, epoxy resins that have been cured with cycloaliphatic amines such as IPDA are generally notable for a high glass transition temperature. Therefore, cycloaliphatic amines are also used especially for the production of composites. Aromatic amines and anhydrides that are likewise used in the production of composites have the disadvantage that long curing times and high curing temperatures are required. Moreover, curing with anhydrides generally leads to comparatively brittle resins. EP 2307358 A states that adding tetramethylguanidine in epoxy resin curing with IPDA and D230 polyetheramine can simultaneously prolong pot life and increase curing rate. However, the systems described therein have comparatively low glass transition temperatures.

Recently, WO 2020/212258A provided amino curing agents which combine fast curing rates typical for conventional aliphatic amino curing agents such as diethylenetriamine (DETA) and long pot-lives and high glass transition temperatures (T_{g}) typical for conventional cycloaliphatic amino curing agents such as isophoronediamine (IPDA). Such amino curing agents are particular suitable for the preparation of fiber-based composites, e.g. per pultrusion, filament winding, prepregs, resin transfer molding (RTM), vacuum aided resin transfer molding (VARTM), bulk mold compression (BMC) or sheet mold compression (SMC).

Against this background, therefore, particularly for the manufacture of composites e.g. by the means of pultrusion, filament winding, fiber impregnation, RTM, VARTM, BMC, or SMC, there is a search for further amino curing agents which, like the curing agents described by WO 2020/212258A combine comparatively long pot life and low mixed viscosity at room temperature (23° C.), and lead to cured epoxy resins having high glass transition temperature and good mechanical properties (such as low brittleness in particular), but simultaneously enable relatively high curing rates at moderate curing temperatures, for example 50 to 120° C., especially 60 to 100° C.

It is an object of the invention, especially for the manufacture of composites, in particular for large composites such wind rotor blades, to provide new amino curing agents and corresponding epoxy resin compositions having an improved curing rate at moderate curing temperatures of 50 to 120° C., especially 60 to 100° C., with simultaneously comparatively long pot life and low mixed viscosity at room temperature. The composition should preferably enable similarly high glass transition temperatures and good mechanical properties (especially low brittleness) for the cured resin to the composition composed of epoxy resin and the cycloaliphatic amino curing agent IPDA.

In the context of this invention, new epoxy resin compositions based on methyl-substituted alkyleneamines as amino curing agents have been identified which combine pot life and viscosity at room temperature that are comparable to epoxy resin compositions based on the cycloaliphatic amino curing agent IPDA, and leads to cured epoxy resins that have similar glass transition temperature and similarly good mechanical properties, but at the same time cure particularly rapidly at a moderate curing temperature of 50 to 120° C., especially 60 to 100° C., and hence are of particularly good suitability for the manufacture of composites, in particular of large composites. The amino curing agents of the invention unexpectedly combine the rapid curing that is typical of aliphatic amines with the relatively long pot lives and relatively high glass transition temperatures that are typical of cycloaliphatic amines.

The present invention accordingly relates to the provision of an aliphatic polyamine which is a compound of formula I,
H₂N (-Q-NH)ₙ-A-NH-Q-NH₂ (I), wherein

n is 0 or 1, and

A is —CH₂—CH(CH₃)—CH₂— or —CH₂—CH₂—CH(CH₃)— or —CH(CH₃)—CH₂—CH₂—, and

each Q is independently —CH(CH₃)—CH₂— or —CH₂—CH(CH₃)—.

In the context of this invention, the term polyamine refers to a compound having at least two primary or secondary amine functions.

In a preferred embodiment, the invention relates to the provision of an aliphatic polyamine of formula I wherein n is 1, the first Q is —CH(CH₃)—CH₂— and the second Q is —CH₂—CH(CH₃)—. This aliphatic polyamine is a compound of formula II,
H₂—CH(CH₃)—CH₂—NH-A-NH—CH₂—CH(CH₃)—NH₂ (II).

With A=—CH₂—CH(CH₃)—CH₂—, this gives the aliphatic polyamine of formula IIa,
H₂N—CH(CH₃)—CH₂—NH—CH₂—CH(CH₃)—CH₂—NH—CH₂—CH(CH₃)—NH₂ (IIa),

and with A=—CH₂—CH₂—CH(CH₃)— or —CH(CH₃)—CH₂—CH₂—, this gives the aliphatic polyamine of formula IIb,
H₂N—CH(CH₃)—CH₂—NH—CH₂—CH₂—CH(CH₃)—NH—CH₂—CH(CH₃)—NH₂ (IIb).

In another preferred embodiment, the invention relates to the provision of an aliphatic polyamine of formula I wherein n is 0 and A is —CH₂—CH(CH₃)—CH₂—. This aliphatic polyamine is a compound of formula III,
H₂N—CH₂—CH(CH₃)—CH₂—NH-Q-NH₂ (III).

With Q=—CH(CH₃)—CH₂—, this gives the aliphatic polyamine of formula IIIa,
H₂—CH₂—CH(CH₃)—CH₂—NH—CH(CH₃)—CH₂—NH₂ (IIIa),

and with Q=—CH₂—CH (CH₃)—, this gives the aliphatic polyamine of formula IIIb,
H₂N—CH₂—CH(CH₃)—CH₂—NH—CH₂—CH(CH₃)—NH₂ (IIIb).

The invention also relates to the provision of any mixtures of two or more different aliphatic polyamines of formula I, particularly the mixtures of the aliphatic polyamines of formula IIa and formula IIb, and the mixtures of the aliphatic polyamines of formula Illa and formula IIIb.

The empiric amine hardener equivalent weight (AHEWₑₘₚ) of the aliphatic polyamines of the invention is preferably in the range from 33 to 40 g/eq, more preferably from 33 to 35 g/eq for the aliphatic polyamines of formula II and in the range from 29 to 35 g/eq, more preferably from 29 to 31 g/eq for the aliphatic polyamines of formula III.

The present invention also relates to an epoxy resin composition comprising at least one epoxy resin and a curing agent component, characterized in that the curing agent component comprises at least one aliphatic polyamine of formula I, particularly at least one aliphatic polyamine of formula II, namely the aliphatic polyamine of formula lla or of formula Ilb or a mixture thereof, or at least one aliphatic polyamine of formula III, namely the aliphatic polyamine of formula IIIa or of formula IIIb or a mixture thereof.

Epoxy resins according to the present invention typically have 2 to 10, preferably 2 to 6, even more preferably 2 to 4, and especially 2 epoxy groups. The epoxy groups are especially glycidyl ether groups as formed in the reaction of alcohol groups with epichlorohydrin. The epoxy resins may be low molecular weight compounds generally having an average molar weight (Mₙ) of less than 1000 g/mol, or higher molecular weight compounds (polymers). Such polymeric epoxy resins preferably have a degree of oligomerization of 2 to 25, more preferably of 2 to 10, units. Said resins may be aliphatic or cycloaliphatic compounds or compounds having aromatic groups. In particular, the epoxy resins are compounds having two aromatic or aliphatic 6-membered rings or oligomers thereof. Epoxy resins of industrial importance are those obtainable by reaction of epichlorohydrin with compounds having at least two reactive hydrogen atoms, especially with polyols. Of particular importance are epoxy resins obtainable by reaction of epichlorohydrin with compounds comprising at least two, preferably two, hydroxy groups and two aromatic or aliphatic 6-membered rings. Such compounds especially include bisphenol A and bisphenol F, and also hydrogenated bisphenol A and bisphenol F, the corresponding epoxy resins being the diglycidyl ethers of bisphenol A or bisphenol F, or of hydrogenated bisphenol A or bisphenol F. The epoxy resin used according to the present invention is typically bisphenol A diglycidyl ether (DGEBA). Suitable epoxy resins according to the present invention also include tetraglycidylmethylenedianiline (TGMDA) and triglycidylaminophenol or mixtures thereof. Also suitable are reaction products of epichlorohydrin with other phenols, for example with cresols or phenol-aldehyde adducts, such as phenol-formaldehyde resins, especially novolacs. Epoxy resins not derived from epichlorohydrin are also suitable. Examples of useful resins include epoxy resins comprising epoxy groups via reaction with glycidyl(meth)acrylate. Preference is given in accordance with the invention to using epoxy resins or mixtures thereof that are liquid at room temperature (23° C.). The epoxy equivalent weight (EEW) gives the average mass of the epoxy resin in g per mole of epoxy group.

The epoxy resin composition of the invention preferably consists to an extent of at least 50% by weight of epoxy resin.

In a particular embodiment, the epoxy resin composition of the invention may additionally comprise reactive diluents. Reactive diluents in the context of the invention are compounds which reduce the mixed viscosity (also initial viscosity) of the epoxy resin composition and which, in the course of the curing of the epoxy resin composition, form a chemical bond with the developing network of epoxy resin and curing agent. Preferred reactive diluents in the context of the present invention are low molecular weight organic, preferably aliphatic, compounds comprising one or more epoxy groups.

Reactive diluents of the invention are preferably selected from the group consisting of butane-1,4-diol diglycidyl ether, hexane-1,6-diol diglycidyl ether (HDDE), glycidyl neodecanoate, glycidyl versatate, 2-ethylhexyl glycidyl ether, neopentyl glycol diglycidyl ether, p-tert-butyl glycidyl ether, butyl glycidyl ether, C8-C10-alkyl glycidyl ethers, C12-C14-alkyl glycidyl ethers, nonylphenyl glycidyl ether, p-tert-butylphenyl glycidyl ether, phenyl glycidyl ether, o-cresyl glycidyl ether, polyoxypropylene glycol diglycidyl ether, trimethylolpropane triglycidyl ether (TMP), glycerol triglycidyl ether, triglycidylparaaminophenol (TGPAP), divinylbenzyl dioxide and dicyclopentadiene diepoxide. They are more preferably selected from the group consisting of butane-1,4-diol diglycidyl ether, hexane-1,6-diol diglycidyl ether (HDDE), 2-ethylhexyl glycidyl ether, C8-C10-alkyl glycidyl ethers, C12-C14-alkyl glycidyl ethers, neopentyl glycol diglycidyl ether, p-tert-butyl glycidyl ether, butyl glycidyl ether, nonylphenyl glycidyl ether, p-tert-butylphenyl glycidyl ether, phenyl glycidyl ether, o-cresyl glycidyl ether, trimethylolpropane triglycidyl ether (TMP), glycerol triglycidyl ether, divinylbenzyl dioxide and dicyclopentadiene diepoxide. They are especially selected from the group consisting of butane-1,4-diol diglycidyl ether, C8-C10-alkyl monoglycidyl ethers, C12-C14-alkyl monoglycidyl ethers, hexane-1,6-diol diglycidyl ether (HDDE), neopentyl glycol diglycidyl ether, trimethylolpropane triglycidyl ether (TMP), glycerol triglycidyl ether and dicyclopentadiene diepoxide.

The reactive diluents of the invention preferably account for a proportion up to 30% by weight, more preferably up to 25% by weight, especially from 1% to 20% by weight, based on the amount of epoxy resin.

The curing agent component of the epoxy resin composition of the invention may also comprise further aliphatic, cycloaliphatic and aromatic polyamines or further primary monoamines. Examples of suitable additional aliphatic, cycloaliphatic or aromatic polyamines include dicycan, dimethyldicycan (DMDC), isophoronediamine (IPDA), diethylenetriamine (DETA), triethylenetetramine (TETA), tetraethylenepentamine (TEPA), 1,3-bis (aminomethyl) cyclohexane (1,3-BAC), bis (p-aminocyclohexyl) methane (PACM), methylenedianiline (for example 4,4′-methylenedianiline), polyetheramines, such as D230 polyetheramine, D400 polyetheramine, D2000 polyetheramine or T403 polyetheramine, 4,9-dioxadodecane-1,12-diamine (DODA), 4,7,10-trioxatridecane-1,13-diamine (TTD), polyaminoamides such as Versamid 140, diaminodiphenylmethane (DDM), diaminodiphenyl sulfone (DDS), toluene-2,4-diamine, toluene-2,6-diamine, 4-methylcyclohexane-1,3-diamine, 2-methylcyclohexane-1,3-diamine, mixtures of 4-methylcyclohexane-1,3-diamine and 2-methylcyclohexane-1,3-diamine (MCDA), 1,2-diaminocyclohexane (DACH), 2,4-diamino-3,5-diethyltoluene, 2,6-diamino-3,5-diethyltoluene (DETDA), 1,2-diaminobenzene, 1,3-diaminobenzene, 1,4-diaminobenzene, diaminodiphenyl oxide, 3,3′,5,5′-tetramethyl-4,4′-diaminodiphenyl, 3,3′-dimethyl-4,4′-diaminodiphenyl, 1,12-diaminododecane, 1,10-diaminodecane, 1,5-diaminopentane (cadaverine), propane-1,2-diamine, propane-1,3-diamine, 2,2′-oxybis (ethylamine), 3,3′-dimethyl-4,4′-diaminodicyclohexylmethane, 4- ethyl-4-methylamino-1-octylamine, ethylenediamine, hexamethylenediamine, menthenediamine, meta-xylylenediamine (MXDA), reaction products of benzene-1,3-dimethanamine with styrene (Gaskamine® 240), N-(2-aminoethyl) piperazine (AEPIP), neopentanediamine, norbornanediamine, dimethylaminopropylaminopropylamine (DMAPAPA), octamethylenediamine, 4,8-diaminotricyclo [5.2.1.0] decane, trimethylhexamethylenediamine, and piperazine. Preferentially suitable as additional aliphatic, cycloaliphatic or aromatic polyamines are dicycan, dimethyldicycan (DMDC), isophoronediamine (IPDA), diethylenetriamine (DETA), triethylenetetramine (TETA), tetraethylenepentamine (TEPA), 1,3-bis(aminomethyl) cyclohexane (1,3-BAC), bis(p-aminocyclohexyl) methane (PACM), polyetheramines, such as D230 polyetheramine, D400 polyetheramine, D2000 polyetheramine or T403 polyetheramine, 4,9-dioxadodecane-1, 12-diamine (DODA), 4,7, 10-trioxatridecane-1,13-diamine (TTD), polyaminoamides such as Versamid 140, 4-methylcyclohexane-1,3-diamine, 2-methylcyclohexane-1,3-diamine, mixtures of 4-methylcyclohexane-1,3-diamine and 2-methylcyclohexane-1,3-diamine (MCDA), 1,2-diaminocyclohexane (DACH), 2,4-diamino-3,5-diethyltoluene, 2,6-diamino-3,5-diethyltoluene (DETDA), 1,5-diaminopentane (cadaverine), meta-xylylenediamine (MXDA), reaction products of benzene-1,3-dimethanamine with styrene (Gaskamine® 240), N-(2-aminoethyl) piperazine (AEPIP) and dimethylaminopropylaminopropylamine (DMAPAPA). Examples of suitable additional primary monoamines include dimethylaminopropylamine (DMAPA) and diethylaminopropylamine (DEAPA).

In a particular embodiment, the aliphatic polyamine of the invention accounts for at least 50% by weight, more preferably at least 80% by weight, most preferably at least 90% by weight, based on the total amount of the curing agents in the epoxy resin composition. In a preferred embodiment, the epoxy resin composition does not comprise any anhydride curing agents. In a particular embodiment, the epoxy resin composition does not comprise any further curing agents aside from the aliphatic polyamine of the invention.

A curing agent in the context of the present invention is understood to mean an amino curing agent or an anhydride curing agent. An amino curing agent in the context of the present invention is understood to mean an amine having an NH functionality of ≥2 (accordingly, for example, a primary monoamine has an NH functionality of 2, a primary diamine has an NH functionality of 4 and an amine having 3 secondary amino groups has an NH functionality of 3). An anhydride curing agent in the context of the present invention is understood to mean an intramolecular carboxylic anhydride, for example 4-methyltetrahydrophthalic anhydride.

In the epoxy resin composition of the invention, preference is given to using the epoxy compounds (epoxy resins including any reactive diluents having epoxy groups) and amino curing agents in an approximately stoichiometric ratio based on the epoxy groups and the NH functionality. Particularly suitable ratios of epoxy groups to NH functionality are, for example, 1:0.8 to 1:1.2. Alternatively, in a particular embodiment of the invention, the epoxy compounds (epoxy resins including any reactive diluents having epoxy groups) and amino curing agents are used in the epoxy resin composition of the invention in an approximately equivalent ratio, preferably in a ratio in the range from 1:0.8 to 1:1.2 based on the EEW of the epoxy compounds and the AHEWₑₘₚ of the amino curing agents.

In a particular embodiment, the epoxy resin composition of the invention may additionally comprise reinforcement fibers. This includes reinforcement fibers which are impregnated with the epoxy resin composition.

The reinforcement fibers of the invention are preferably glass fibers, carbon fibers, aramid fibers or basalt fibers, or mixtures thereof. Particular preference is given to glass fibers and carbon fibers, especially glass fibers. Glass fibers used are typically fibers of E glass, but also those of R glass, S glass and T glass. The choice of glass type can influence the mechanical properties of the composite materials. According to the invention, the reinforcing fibers are used in the form of single fibers, but preferably in the form of fiber filaments, fiber rovings, fiber mats or combinations thereof. Particular preference is given to using the reinforcing fibers in the form of fiber rovings. The reinforcing fibers may take the form, for example, of short fiber sections having a length of a few mm to cm or of mid-length fiber sections having a length of a few cm to a few m or of long fiber sections having a length in the range of a few m or more. According to the invention, reinforcing fibers are preferably used in the form of continuous fiber filaments, continuous fiber rovings or continuous fiber mats, especially for the use in pultrusion or filament winding. Continuous fiber filaments, continuous fiber rovings or continuous fiber mats in the context of the invention have a length of at least 10 m, preferably of at least 100 m, especially of at least 200 m.

The epoxy resin composition of the invention may also comprise further additives, for example inert diluents, curing accelerators, pigments, colorants, fillers, release agents, tougheners, flow agents, antifoams, flame retardants or thickeners. Such additives are typically added in functional amounts, for example, a pigment is typically added in an amount that leads to the desired color for the composition. The compositions of the invention typically comprise from 0% to 50% by weight, preferably 0% to 20% by weight, for example 2% to 20% by weight, for the entirety of all additives based on the epoxy resin composition. In the context of the present invention, additives are understood to mean all additions to the epoxy resin composition that are neither epoxy compound nor curing agent (amino curing agent and/or anhydride curing agent) nor reinforcing fiber.

The invention further provides the use of the aliphatic polyamine of the invention for the curing of an epoxy resin.

The invention further provides a method of producing cured materials form the epoxy resin composition of the invention. In such method the epoxy resin composition of the invention is provided and then cured. For this purpose, the components of the epoxy resin composition are contacted with one another and then are cured at a temperature practicable for use. Usually for cured composite materials, first the epoxy compounds, the curing agents and the additives, if any, of the epoxy resin composition are contacted with one another and mixed, are subsequently contacted (impregnation or embedding) with the reinforcing fibers, and then are cured at a temperature practicable for use. The curing is preferably effected at a temperature of at least 50° C., more preferably of at least 60° C. The curing can be effected at temperatures of less than 120° C., especially at temperatures of less than 100° C., especially within a temperature range from 50 to 120° C., most preferably within a temperature range from 60 to 120° C. The curing can preferably be effected under standard pressure. The production processes for cured composite materials include the curing of pre-impregnated fibers or fiber weaves (e.g. prepregs curing, filament winding method or pultrusion method), and the production of composite moldings by means of infusion or injection methods such as vacuum-assisted resin transfer molding (VARTM), resin transfer molding (RTM), and also wet compression methods such as BMC (bulk mold compression) and SMC (sheet mold compression).

The invention further provides the cured material obtainable or obtained by curing the epoxy resin composition of the invention, e.g. the cured composite material obtainable or obtained by curing an epoxy resin composition of the invention comprising reinforcement fibers. More particularly, the invention provides cured material or cured composite material obtainable or obtained by the method of the invention for producing cured material or cured composite material, respectively. The cured materials, e.g the cured composite materials, cured in accordance with the invention have a comparatively high glass transition temperature T_{g}.

By means of the methods of the invention, especially by means of the pultrusion method of the invention and by means of the filament winding method of the invention, it is possible to produce rebars. Such rebars are particularly weathering-resistant, whereas conventional rebars made of steel are subject to corrosion. The use of such rebars in concrete structures therefore enables the building of particularly long-lived structures. Such rebars can be produced in any length and thickness; the rebars preferably have lengths in the range from 0.5 to 50 m, especially from 1 to 20 m, and thicknesses of 0.5 to 5 cm, especially of 1 to 3 cm. The cross section of such rebars may have any geometry; it is preferably essentially rectangular or circular. Such rebars preferably have a surface profile, for example one or more grooves or elevations forming a spiral around the rebar, in order to improve securing within the concrete. Such surface profiles can, for example, be machined subsequently into the already cured rebar, or be applied by wrapping with corresponding impregnated reinforcement fiber material prior to curing. Such rebars may also have an additional surface coating, for example of further epoxy resin composition, in order to additionally protect the reinforcing fibers from weathering, and from chemical and thermal influences, or in order to improve interaction with the concrete.

The invention further provides a method of producing the aliphatic polyamine of formula IIa, characterized in that in a first step methacrolein and propane-1,2-diamine are reacted according to the below reaction scheme

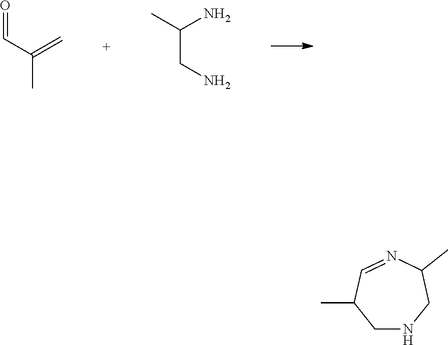

to form the cyclic intermediate compound of formula IV,

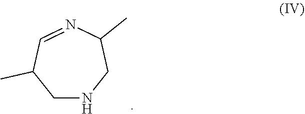

In a second step this cyclic intermediate compound of formula IV is reacted with further propane-1,2-diamine in the presence of hydrogen and a hydrogenation catalyst according to the below reaction scheme to form the aliphatic polyamine of formula IIa.

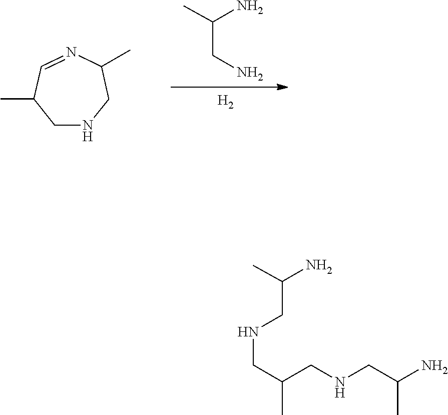

In the first step, usually the propane-1,2-diamine is used in a molar excess relative to methacroleine, usually in the range of 1.5 to 10-fold, preferably in the range of 3 to 8-fold. Preferred reaction temperature for the first step is in the range of 10 to 70° C., more preferably in the range of 20 to 50° C. The reaction mixture of the first step can be used for the second step without any purification step. In the second step, usually the propane-1,2-diamine is used in a molar excess relative to cyclic intermediate compound of formula IV, usually in the range of 1.5 to 9-fold, preferably in the range of 3 to 7-fold. For the second step a suitable amount of a hydrogenation catalyst, preferably a heterogeneous hydrogenation catalyst is added to the reaction mixture. Suitable hydrogenation catalysts are based on Co, Ni, Pt, Ru, Rh, Pd and mixtures thereof. The catalytically active metals can be used in elemental form (such as Raney-Cobalt or Raney-Nickel), or in their oxidized form (as Oxides, Chlorides, Nitrates, such as PtO₂ (Adams Catalyst)) and can be supported on a solid support selected from Al₂O₃, ZrO₂, TiO₂, SiO₂, activated carbon and mixtures thereof (such as Ru/C or Co/Al₂O₃). Both fixed-bed catalysts and suspension catalysts can be used. Particularly preferred is the use of a hydrogenation catalyst having Pd as catalytically active metal on a support of activated carbon (“Pd/C”). The hydrogen for the hydrogenation is usually applied with a pressure in the range of 10 to 200 bar, preferably in the range of 20 to 100 bar. Preferred reaction temperature for the first step is in the range of 50 to 100° C., more preferably in the range of 60 to 90° C. The resulting aliphatic polyamine of formula lla can be purified by the means of fractionated distillation, preferably after filtering off the catalyst and evaporating remaining excess of propane-1,2-diamine.

The invention further provides a method of producing the aliphatic polyamine of formula IIb, characterized in that in a first step methyl vinyl ketone and propane-1,2-diamine are reacted according to the below reaction scheme

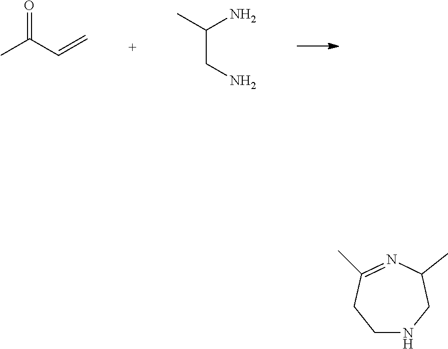

to form the cyclic intermediate compound of formula V,

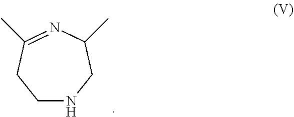

In a second step this cyclic intermediate compound of formula V is reacted with further propane-1,2-diamine in the presence of hydrogen and a hydrogenation catalyst according to the below reaction scheme to form the aliphatic polyamine of formula IIb.

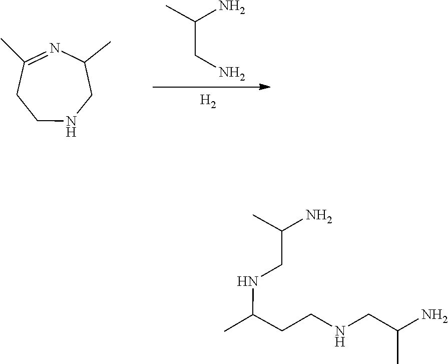

In the first step, usually the propane-1,2-diamine is used in a molar excess relative to methyl vinyl ketone, usually in the range of 1.5 to 10-fold, preferably in the range of 3 to 8-fold. Preferred reaction temperature for the first step is in the range of 10 to 70° C., more preferably in the range of 20 to 50° C. The reaction mixture of the first step can be used for the second step without any purification step. In the second step, usually the propane-1,2-diamine is used in a molar excess relative to cyclic intermediate compound of formula V, usually in the range of 1.5 to 9-fold, preferably in the range of 3 to 7-fold. For the second step a suitable amount of a hydrogenation catalyst, preferably a heterogeneous hydrogenation catalyst is added to the reaction mixture. Suitable hydrogenation catalysts are based on Co, Ni, Pt, Ru, Rh, Pd and mixtures thereof. The catalytically active metals can be used in elemental form (such as Raney-Cobalt or Raney-Nickel), or in their oxidized form (as Oxides, Chlorides, Nitrates, such as PtO₂ (Adams Catalyst)) and can be supported on a solid support selected from Al₂O₃, ZrO₂, TiO₂, SiO₂, activated carbon and mixtures thereof (such as Ru/C or Co/Al₂O₃). Both fixed-bed catalysts and suspension catalysts can be used. Particularly preferred is the use of a hydrogenation catalyst having Pd as catalytically active metal on a support of activated carbon (“Pd/C”). The hydrogen for the hydrogenation is usually applied with a pressure in the range of 10 to 200 bar, preferably in the range of 20 to 100 bar. Preferred reaction temperature for the first step is in the range of 50 to 100° C., more preferably in the range of 60 to 90° C. The resulting aliphatic polyamine of formula IIb can be purified by the means of fractionated distillation, preferably after filtering off the catalyst and evaporating remaining excess of propane-1,2-diamine.

The invention further provides a method of producing the aliphatic polyamines of formula Illa and IIIb, characterized in that in a first step methacrylonitrile and propane-1,2-diamine are reacted according to the below reaction scheme

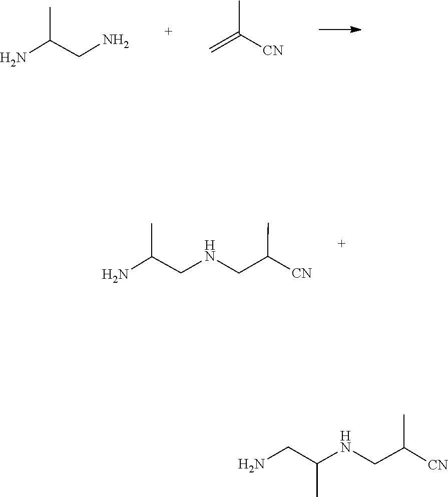

to form the intermediate nitrile compounds of formula VI,

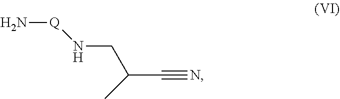

with Q=—CH(CH₃)—CH₂— or —CH₂—CH(CH₃)—.

In a second step these intermediate nitrile compounds of formula VI ate hydrogenated in the presence of hydrogen and a hydrogenation catalyst according to the below reaction scheme

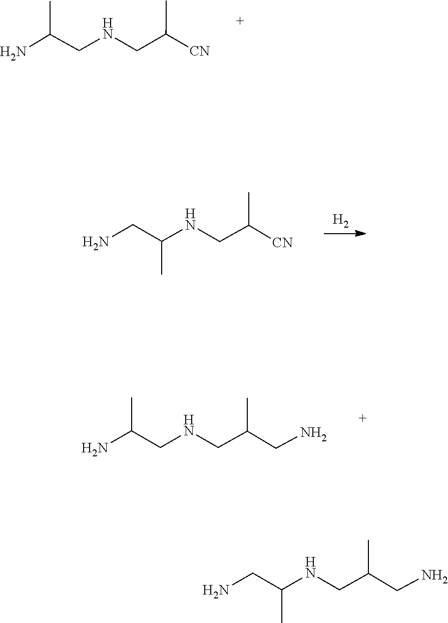

to form the aliphatic polyamines of formula Illa and Illb. In the first step, the propane-1,2-diamine is usually used in a molar excess relative to methacrylonitrile, preferably in the range of 1.5 to 8-fold, more preferably in the range of 2 to 5-fold. The reaction of the first step is usually performed under a pressure in the range of 5 to 200 bar, preferably in the range of 10 to 50 bar using gases which are inert under the given conditions, such as N₂, Ar or H₂, or mixtures thereof. Preferred reaction temperature for the first step is in the range of 120 to 220° C., more preferably in the range of 150 to 200° C. Before starting the second step the remaining excess of propane-1,2-diamine and other low boiling compounds are preferably removed from the intermediate nitril compounds of formula VI by the means of distillation, preferably under reduced pressure. For the second step a suitable amount of a hydrogenation catalyst, preferably a heterogeneous hydrogenation catalyst is added to the reaction mixture. Suitable hydrogenation catalysts are based on Co, Ni, Pt, Ru, Rh, Pd and mixtures thereof. The catalytically active metals can be used in elemental form (such as Raney-Cobalt or Raney-Nickel), or in their oxidized form (as Oxides, Chlorides, Nitrates, such as PtO₂ (Adams Catalyst)) and can be supported on a solid support selected from Al₂O₃, ZrO₂, TiO₂, SiO₂, activated carbon and mixtures thereof (such as Ru/C or Co/Al₂O₃). Both fixed-bed catalysts and suspension catalysts can be used. The hydrogen for the hydrogenation is usually applied with a pressure in the range of 50 to 300 bar, preferably in the range of 100 to 200 bar. Preferred reaction temperature for the second step is in the range of 60 to 150° C., more preferably in the range of 80 to 120° C. Preferably the second step is carried out in the presence of ammonia, preferably in a molar excess relative to the intermediate nitril compounds of formula VI, usually in the range of 2 to 20-fold, preferably in the range of 2 to 15-fold. The resulting aliphatic polyamines of formula IIIa and Illb can be purified by the means of fractionated distillation, preferably after filtering off the catalyst.

The gel time according to standard DIN 16 945 (1989) gives an indication as to the period of time between the addition of the curing agent to the reaction mixture and the transition of the reactive resin composition from the liquid state to the gel state. The temperature plays an important role, and the gel time is therefore determined for a predetermined temperature in each case. Dynamic-mechanical methods, in particular rotational viscometry, make it possible to analyze even small sample quantities in quasi-isothermal fashion and to capture their entire viscosity/stiffness profile. According to standard ASTM D 4473-08 (2016), the point of intersection between the storage modulus G′ and the loss modulus G″ at which the damping tan δ has a value of 1 is the gel point, and the period of time between addition of the curing agent to the reaction mixture and attainment of the gel point is the gel time. The gel time thus determined at elevated temperature (e.g. 90 or 110° C.) can be regarded as a measure of the curing rate at such elevated temperature while the gel time thus determined at room temperature (23° C.) can be regarded as a measure of the handling time at such ambient temperature.

For determination of the B time, which likewise serves as a measure of the curing rate, according to standard DIN EN ISO 8987 (2005), a sample (for example 0.5 g) of the freshly produced reactive resin composition is applied to a hot plate (for example an unrecessed plate, for example at 145° C.), and the time until formation of threads (gel point) or until abrupt hardening (curing) is determined.

The glass transition temperature (T_{g}) can be determined using a differential calorimeter (DSC), for example in accordance with standard ASTM D 3418-15 (2015). This involves heating a very small amount of sample (for example about 10 mg) in an aluminum crucible (for example at 20° C./min) and measuring the heat flow to a reference crucible. This cycle is repeated three times. The glass transition is determined from the second measurement or as the average of the second and third measurements. The evaluation of the T_{g} step of the heat-flow curve can be determined via the inflection point, according to the half width or according to the midpoint temperature method.

The amine hydrogen equivalent weight (AHEW) can be determined either theoretically or empirically, as described by B. Burton et al (Huntsman, “Epoxy Formulations using Jeffamine Polyetheramines”, Apr. 27, 2005, p. 8-11). The theoretically calculated AHEW is defined as the quotient of the molecular weight of the amine divided by the number of available amine hydrogens (for example 2 for every primary amino group plus 1 for every secondary amino group). For IPDA, for example, having a molecular weight of 170.3 g/mol and 2 primary amino groups, i.e. 4 available amine hydrogens, the theoretically calculated AHEW is 170.3/4 g/eq=42.6 g/eq. The determination of the empirical AHEW is based on the assumption that equivalent amounts of epoxy resin and amino curing agent result in a cured epoxy resin characterized by a maximum heat distortion resistance (heat distortion temperature (HDT)) or maximum gas transition temperature (T_{g}). Therefore, in order to ascertain the empirical AHEW, mixtures of a fixed amount of epoxy resin and a varying amount of the amino curing agent are cured as completely as possible, the respective HDT or T_{g} thereof is determined, and the characteristics thus ascertained are plotted against the ratio of the starting materials. The empirical AHEW (AHEWₑₘₚ) is defined by the following formula:

$$A H E {W}_{e m p} = ( {AH}_{max} * EE {W}_{epox} ) / ER$$

with AHₘₐₓ=amount of the amino curing agent at maximum HDT or T_{g} in grams EEWₑₚₒₓ=EEW value of the epoxy resin used for the test
   ER=amount of the epoxy resin used for the test in grams

In the context of this invention, AHEWemp means an empirical amine hydrogen equivalent weight based on the determination of a maximum T_{g} (measured by means of DSC according to standard ASTM D 3418-15 (2015)). The empirical AHEWₑₘₚ is of particular significance in cases where the theoretically calculated AHEW is unobtainable, for example in the case of mixtures of polymeric amines.

The initial viscosity (“mixed viscosity”) of a curable composition, for example the matrix component of the fiber-matrix composition of the invention, can be determined according to standard DIN ISO 3219 (1993) directly after the mixing of the constituents. The mixed viscosity is with the aid of a shear stress-controlled rheometer (e.g. MCR 301 from Anton Paar) with cone-plate arrangement (for example diameter of cone and plate: 50 mm; cone angle: 1°; gap width: 0.1 mm). The measurement temperature has a major influence on the viscosity and curing rate of the curable composition and is therefore a crucial factor in these measurements. Accordingly, the mixed viscosity must be determined at a particular temperature, for example at room temperature (23° C.), in order to be comparable.

The impact resistance of a test specimen composed of cured epoxy resin can be determined by means of the Charpy notched bar impact test according to standard DIN EN ISO 179-1 (2010) at room temperature. High impact resistance corresponds to low brittleness.

### EXAMPLES

### Example 1a

### Synthesis of an Aliphatic Polyamine of Formula IIa

In a first step an excess of 491.4 g (6.56 mol) propane-1,2-diamine were added to 100.0 g (1.31 mol) of methacroleine within 90 minutes at room temperature. An ice bath was used to keep the temperature constant in a range of 25 to 30° C. The mixture was stirred for one hour. This reaction step resulted in the formation of the cyclic intermediate compound of formula IV. The formation of this intermediate compound based on a 7-membered ring was verified by GC-MS analysis.

In a second step the reaction mixture of the first step, still containing an excess of the propane-1,2-diamine, has been transferred to an autoclave. 20 g of Pd/C hydrogenation catalyst (5% Pd on activated carbon, Sigma Aldrich) were added. Subsequently, 10 bar of hydrogen were applied, then the autoclave was heated to 80° C. within 15 minutes and finally 50 bar of hydrogen were applied. This mixture was stirred at a temperature of 80° C. for 24 hours, resulting in the formation of the aliphatic polyamine of formula IIa.

The catalyst was filtered off, the excess propane-1,2-diamine was evaporated on a rotavap at a temperature of 90° C. and the residue was subjected to a distillation.

The above-mentioned reaction was repeated and both samples were combined for the distillation. Using a reflux ratio between 5:2 and 5:1 the distillation was carried out at a reduced pressure in the range of 1.7 to 2.1 mbara. 244.5 g (1.21 mol, 46% yield, purity >99% GC) of the aliphatic polyamine of formula lla in the form of a colorless liquid with a boiling point of 117 to 119° C. (at 2.1 mbara) were obtained. NMR analysis of the final product revealed a stereo isomeric mixture.

### Example 1b

### Synthesis of an Aliphatic Polyamine of Formula IIb

In a first step an excess of 467.3 g (6.24 mol) propane-1,2-diamine were added to 100.0 g (1.25 mol) of methylvinylketone within 53 minutes at room temperature. An ice bath was used to keep the temperature constant in a range of 21 to 30° C. The mixture was stirred for one hour. This reaction step resulted in the formation of the cyclic intermediate compound of formula V. The formation of this intermediate compound based on a 7-membered ring was verified by GC-MS analysis. This reaction step was carried out twice.

In a second step 56.7 g of the above reaction mixture of the first step, still containing an excess of the propane-1,2-diamine, has been transferred to a 150 ml autoclave. 2 g of Pd/C hydrogenation catalyst (5% Pd on activated carbon, Sigma Aldrich) were added. Subsequently, 10 bar of hydrogen were applied, then the autoclave was heated to 80° C. within 15 minutes and finally 50 bar of hydrogen were applied. This mixture was stirred at a temperature of 80° C. for 12 hours, resulting in the formation of the aliphatic polyamine of formula IIb.

The catalyst was filtered off, the excess propane-1,2-diamine was evaporated on a rotavap at a temperature of 60° C. and the residue was subjected to a distillation.

The above-mentioned second step was carried out twenty times in total and all samples were combined for the distillation.

Using a reflux ratio between 5:2 and 5:1 the distillation was carried out at a reduced pressure of 1.8 mbara. 33 g (purity >98% GC) of the aliphatic polyamine of formula IIb in the form of a yellow liquid with a boiling point of 108.6° C. (at 1.8 mbara) were obtained. NMR analysis of the final product revealed a stereo isomeric mixture.

### Example 1c

### Synthesis of an Aliphatic Polyamine Mixture of Formula IIIa and IIIb

An autoclave was charged with a mixture of methacrylonitrile (30 g, 0.45 mol) and propane-1,2-diamine (100 g, 1.35 mol). The autoclave was sealed, pressurized with H₂ to 20 bar and heated to 170° C. within 3 h. The mixture was stirred at 170° C. over night, cooled to ambient temperature and depressurized. The crude mixture contained ˜50% propane-1,2-diamine, ˜35% of a mixture of 3-((2-aminopropyl)amino)-2-methylpropanenitrile and 3-((1-aminopropan-2-yl)amino)-2-methylpropanenitrile, and ˜5% of other compounds according to GC (values in GC-Area-%).

Five identical batches of crude reaction mixture were pooled, and propane-1,2-diamine was removed by distillation along with other light-boilers under reduced pressure. The distillation sump (p=20 mbara, T_{sump.max}=85° C.) contained ˜2% propane-1,2-diamine, ˜89% of a mixture of 3-((2-aminopropyl)amino)-2- methylpropanenitrile and 3-((1-aminopropan-2-yl)amino)-2-methylpropanenitrile, and ˜9% of other compounds according to GC (values in GC-Area-%). The mixture was used as such in the subsequent hydrogenation reaction.

An autoclave was charged with the crude sump product of the previous reaction step (50 g), and g of Raney-Cobalt (washed with THF to remove water). The autoclave was sealed, pressurized with H₂ to 10 bar and NH₃ (40 g, 2.4 mol) was added. The reaction mixture was heated to 100° C. and pressurized with H₂ to 170 bar. After a reaction time of 8 h, the mixture was cooled to ambient temperature, depressurized and Raney-Cobalt was removed by filtration. The crude mixture contained ˜2% propylene-1,2-diamine, ˜90% of a mixture of N1-(3-amino-2-methylpropyl)propane-1,2-diamine (compound of formula IIIb) and N2-(3-amino-2-methylpropyl)propane-1,2-diamine (compound of formula IIIa) and 8% of other compounds according to GC (values in GC-Area-%, residual THF not included).

Four identical batches of crude reaction mixture were pooled and purified by distillation at 2 mbara. A mixture of of 3-((2-aminopropyl)amino)-2-methylpropanenitrile and 3-((1-aminopropan-2-yl)amino)-2-methylpropanenitrile (113 g) was obtained at T_{head}=58 to 62° C. The identity of the obtained compounds was confirmed by GC-MS with a molecular mass of 145 g.

### Example 2: Curing of Epoxy Resin With the Aliphatic Polyamines

The aliphatic polyamines from example 1a, 1b or 1c and epoxy resin (bisphenol A diglycidyl ether, Epilox A19-03, Leuna, EEW: 185 g/mol) according to the amounts stated in table 1 were mixed in a stirrer system (1 min at 2000 rpm). DSC measurements (differential scanning calorimetry) and rheological analyses were performed immediately after mixing. By way of comparison, corresponding compositions comprising IPDA (Baxxodur® EC 201, BASF), diethylenetriamine (DETA, BASF), dimethyldiethylenetriamine (DMDETA; prepared according to example 1a of WO 2020/212258 A), and tetramethyltrieethyltetramine (TMTETA; with the preparation of DMDETA according to example 1a of WO 2020/212258 A also a lower amount of the corresponding TMTETA is formed which is isolated from the reaction mixture by fractionated distillation) were also examined in the same way.

The DSC analyses of the curing reaction of these mixtures for determination of onset temperature (Tₒ), exothermic enthalpy (ΔH) and glass transition temperature (T_{g}) were conducted according to ASTM D 3418-15 (2015), using the following temperature profile: 0° C.→20 K/min 200° C.→10 min 200° C. The T_{g} was determined in the second run. The results are collated in table 1.

The rheological measurements for examination of the reactivity profile (pot life and gel time) of the various amino curing agents with the epoxy resin were conducted at different temperatures on a shear stress-controlled plate-plate rheometer (MCR 301, Anton Paar) with a plate diameter of mm and a gap of 0.25 mm. The gel times were determined with oscillation of the abovementioned rheometer at 23° C., 70° C., 90° C. or 110° C., with the point of intersection of the loss modulus (G″) and storage modulus (G′) giving the gel time according to standard ASTM D 4473-08 (2016). The gel time at 23° C. serves as a measure for the handling time at room temperature while the gel time at 70° C., 90° C. or 110°° C. serves as a measure for the curing rate at elevated temperature. The mixed viscosities (ηₒ) were measured at room temperature (23° C.) according to standard DIN ISO 3219 (1993) immediately after the components had been mixed, with the aid of a shear stress-controlled rheometer (e.g. MCR 301 from Anton Paar) with cone-plate arrangement (e.g. diameter of cone and plate: 50 mm; cone angle: 1°; gap width: 0.1 mm) For the determination of the B times that likewise serve as a measure of curing rate, samples (about 0.5 g) of the freshly produced reactive resin composition were applied to an unrecessed plate at 145° C. and, according to standard DIN EN ISO 8987 (2005), the time taken to form fibers (gel point) and until abrupt hardening (curing) were determined. The results of the rheological measurements are summarized in table 1.

Immediately after the epoxy resin and amino curing agent system had been mixed, the mixture was degassed at 1 mbar and then cured (8 h at 60° C., then 4 h at 100° C., then 2 h at 160° C.). After curing, the mechanical properties for the cured resin (tensile modulus of elasticity (E-t), tensile strength (σ-M), tensile elongation (ε-M), flexural modulus of elasticity (E-f), flexural strength (σ-fM) and flexural elongation (ε-fM)) were determined at room temperature according to standards ISO 527-2:1993 and ISO 178:2006. The results are likewise collated in table 1. Impact resistance was determined by means of the Charpy notched bar impact test according to standard DIN EN ISO 179-1 (2010) at room temperature. High impact resistance corresponds to low brittleness.

TABLE 1
Comparison of the curing of epoxy resin with various amino curing
agents (inventive: aliphatic polyamines from example 1a, 1b or
1c; comparative experiments: IPDA, DETA, DMDETA and TMTETA)

| | | | | DM- | TM- | | |
|---|---|---|---|---|---|---|---|
| | Ex. 1a | Ex. 1b | Ex. 1c | DETA | TETA | IPDA | DETA |
| AHEWₑₘₚ | 34.2 | 34.2 | 30.5 | 27 | 31.5 | 43 | 20.6 |
| Amount of curing agent (g) | 18.5 | 18.5 | 16.5 | 14.6 | 17.0 | 23.2 | 11.1 |
| per 100 g of epoxy resin | | | | | | | |
| ηₒ (mPas) at 23° C. | 1900 | 2115 | 1185 | 1435 | 2230 | 1930 | 1540 |
| Gel time (min) at 23° C. | 625 | 605 | 440 | 514 | 636 | 470 | 280 |
| Gel time (min) at 70° C. | 39 | 39 | 31 | 39 | 52 | 44 | 15 |
| Gel time (min) at 90° C. | 11.8 | 11.7 | 9.0 | 11.0 | 13 | 18.0 | 6.0 |
| Gel time (min) at 110° C. | 4.8 | 5.1 | 4.2 | 4.6 | 5.5 | 7.5 | 2.6 |
| B time of plate (sec) | 65 | 60 | 60 | 80 | 70 | 130 | 25 |
| at 145° C. (gel point) | | | | | | | |
| B time of plate (sec) | 75 | 70 | 70 | 90 | 80 | 145 | 30 |
| at 145° C. (curing) | | | | | | | |
| Tₒ (° C.) | 70 | 68 | 65 | 66 | 67 | 68 | 62 |
| ΔH (J/g) | 474 | 464 | 515 | 487 | 432 | 467 | 540 |
| T_{g} (° C.) | 161.8 | 161.8 | 150.0 | 161.5 | 165.8 | 164.9 | 136.7 |
| Flexural E-f (MPa) | 2959 | n.d. | n.d. | 3057 | 3232 | 2884 | 2865 |
| Flexural σ-fM (MPa) | 112 | n.d. | n.d. | 116.3 | 132 | 119.4 | 104 |
| Flexural ε- fM (%) | 5.7 | n.d. | n.d. | 6.09 | 6.1 | 6.08 | 5.9 |
| Tensile E-t (MPa) | 2914 | n.d. | n.d. | 2938 | 2960 | 2734 | 2726 |
| Tensile σ-M (MPa) | 78.8 | n.d. | n.d. | 81 | 76 | 80.1 | 70.6 |
| Tensile ε-M (%) | 6.5 | n.d. | n.d. | 8.6 | 5.1 | 7.8 | 6.2 |
| Charpy (kJ/m²) | 18.9 | n.d. | n.d. | 26 | 25 | 38.9 | 22.6 |

(n.d.: not determined)

1. An aliphatic polyamine of the formula I,
H₂N(-Q-NH)ₙ-A-NH-Q-NH₂ (I),
wherein n is 0 or 1,
A is —CH₂—CH(CH₃)—CH₂—, —CH₂—CH₂—CH(CH₃)—, or —CH(CH₃)—CH₂—CH₂—, and
each Q is independently —CH(CH₃)—CH₂—or —CH₂—CH(CH₃)—.

2. The aliphatic polyamine of claim 1, wherein n and Q are selected such that the aliphatic polyamine is a compound of formula II,
H₂N—CH(CH₃)—CH₂—NH—A-NH—CH₂—CH(CH₃)—NH₂ (II).

3. The aliphatic polyamine of claim 2, wherein A is —CH₂—CH(CH₃)—CH₂—.

4. The aliphatic polyamine of claim 2, wherein A is —CH₂—CH₂CH(CH₃)— or —CH(CH₃)—CH₂—CH₂—.

5. The aliphatic polyamine of claim 1, wherein n is 0 and A is —CH₂—CH(CH₃)—CH₂—.

6. An epoxy resin system, comprising:
at least one epoxy resin component, and
a curing agent component,
wherein the curing agent component comprises at least one aliphatic polyamine according to claim 1.

7. The epoxy resin system of claim 6, wherein the composition additionally comprises reinforcement fibers.

8. (canceled)

9. A method of producing cured materials, comprising:
providing mixing the epoxy resin component and curing agent component according to claim 6, and
curing the epoxy resin system.

10. A cured material, obtained by the method according to claim 9.

11. A method of producing the aliphatic polyamine according to claim 3, comprising:
first, reacting methacrolein and propane-1,2-diamine to form a cyclic compound of formula IV, 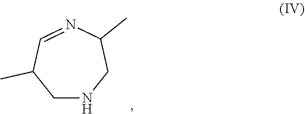
and second, reacting the cyclic compound of formula IV with further propane-1,2-diamine in the presence of hydrogen and a hydrogenation catalyst to form the aliphatic polyamine.

12. A method of producing the aliphatic poly amine according to claim 4, comprising:
first, reacting methyl vinyl ketone and propane-1,2-diamine to form a cyclic compound of formula V, 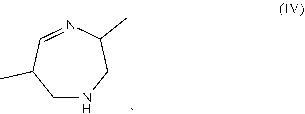
and second, reacting the cyclic compound of formula V with further propane-1,2-diamine in the presence of hydrogen and a hydrogenation catalyst to form the aliphatic polyamine.

13. A method of producing the aliphatic polyamine according to claim 5, comprising:
first, reacting methacrylonitrile and propane-1,2-diamine to form a nitrile of formula VI, 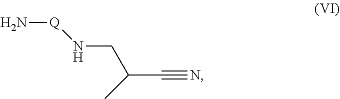
with Q=—CH(CH₃)—CH₂—or —CH₂—CH(CH₃)—,
and second, hydrogenating the nitrile of formula VI in the presence of hydrogen and a hydrogenation catalyst to form the aliphatic polyamine.
